(19) United States
(12) Patent Application Publication
(10) Pub. No.: US 2021/0353797 A1
(43) Pub. Date: Nov. 18, 2021

(54) Ultraviolet light tower

(71) Applicant: Nemesis UVC LLC, Garland, TX (US)

(72) Inventors: Michael Nevitt, Garland, TX (US); Eric L. Wade, Garland, TX (US); John Early, Garland, TX (US)

(21) Appl. No.: 17/318,628

(22) Filed: May 12, 2021

Related U.S. Application Data
(60) Provisional application No. 63/023,658, filed on May 12, 2020.

(51) Int. Cl. A61L 2/10

(52) U.S. Cl. CPC A61L 2/10; A61L 2202/11; A61L 2202/16; A61L 2202/25; A61L 2202/14

(57) ABSTRACT

A disinfecting tower rack, preferably a UV light tower having a base, an upper plate, and a main body separating the base and the upper plate, thus defining an overall height. The UV light tower main body is configured to receive and hold or secure to one or more UV light panels. Each UV light panel may be operationally separate and distinct from adjacent or other UV light panels and are designed to work independently, allowing each panel to be removed without impacting the functioning of any of the other secured or intact UV light panels.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/023,658 entitled “ULTRAVIOLET LIGHT TOWER” filed May 12, 2020. The contents of the above referenced application are incorporated herein by reference in its entirety.

### FIELD OF THE INVENTION

The present invent ion relates to devices, systems, and methods for disinfecting an area; to devices and methods for housing one or more ultraviolet (UV) lights or panels to prevent, microbial or pathogen growth within an area; and more particularly, to a portable ultraviolet (UV) light tower for use in preventing microbial or pathogen growth within an area which can be easily assembled and disassembled.

### BACKGROUND OF THE INVENTION

The worldwide 2020 Coronavirus pandemic emergency illustrates the need for preventing or inhibiting microbial growth, such as the virus responsible for COVID 19, to prevent disease. While it is nearly impossible to maintain a strategy of preventing all microbial growth in every aspect of society, the need to quickly and easily provide devices and systems to do so is more important than ever. Use of ultraviolet (UV) light is a well known technique for killing many types of microbes. Such lighting systems have been used as extreme cleaning techniques where sanitation and disinfection are critical. Such extreme sanitization techniques are typically utilized in areas that need it most, such as hospitals. As the “shelter in place” policy that most countries adapted to minimize exposure to the virus begins to end, more individuals will be returning to daily life where they will be exposed to many surfaces, areas, and crowds. These interactions greatly increase exposure to the disease.

Typical UV light systems used in hospital settings are either permanently fixed to a room or utilize movable units that are large, expensive, and not necessarily designed to travel from one place to another outside a hospital setting, such as a patient room or surgical suite. UV light systems that are mobile and can be assembled or disassembled quickly and easily are needed.

### SUMMARY OF THE INVENTION

Embodied are devices, systems, and methods for disinfecting an area using one or more ultraviolet (UV) lights or panels. The devices include, a portable ultraviolet (UV) tower which includes removable ultraviolet (UV) light panels. The devices are easily transportable and can be quickly assembled and disassembled.

Accordingly, it is an objective of the invention to provide a unique disinfection system.

It is a further objective of the invention to provide a disinfection system using UV light.

It is a further objective of the invention to provide a 360-degree disinfection system using UV light.

It is yet another objective of the invention to provide a unique disinfection system using UV light that is portable.

It is a still further objective of the invention to provide a disinfection system using UV light that can be easily moved from one area to another.

It is a further objective of the invention to provide a disinfection system using UV light that can foe easily assembled.

It is yet another objective of the invention to provide a disinfection system using UV light that can be easily disassembled.

It is a still further objective of the invention to provide a disinfection system using one or more UV light panels.

It is a further objective of the invention to provide a disinfection system using one or more removable UV light panels.

It is yet another objective of the invention to provide a disinfection system using one or more removable UV light panels which can be stored and/or transported in a case.

It is a still further objective of the invention to provide a disinfection system using UV light having an in-use warning system.

It is a further objective of the invention to provide a disinfection system using UV light utilizing an LED beacon in-use warning light.

Other objectives and advantages of this invention will become apparent from the following description taken in conjunction with any accompanying drawings wherein are set forth by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a perspective view of an illustrative embodiment of a disinfecting tower rack, shown with UV lights;

FIG. 2 is a side perspective view of the disinfecting tower rack illustrated in FIG. 1;

FIG. 3 is an alternative perspective view of the disinfecting tower rack;

FIG. 4 is a top perspective view of the disinfecting tower rack;

FIG. 5 illustrates the disinfecting tower rack placed within a locker room, illustrating the 360 degree UV light exposure within the locker room;

FIG. 6 illustrates the disinfecting tower rack with the UV light panels removed;

FIG. 7 illustrates an embodiment of an inner frame;

FIG. 8 is a perspective view of an illustrative embodiment of a base component;

FIG. 9 illustrates an embodiment of a lower plate of the base component;

FIG. 10 illustrates an embodiment of an upper plate of the base component;

FIG. 11 illustrates the base component with a side wall removed;

FIG. 12 illustrates a smaller plate, which secures to the upper plate of the base component;

FIG. 13 illustrates an embodiment of the upper plate;

FIG. 14 is an alternative view of the upper plate;

FIG. 15 illustrates an example of a UV LSD light panel;

FIG. 16A is an illustrative embodiment of a disinfecting tower rack travel case;

FIG. 16B is an illustrative embodiment of a disinfecting tower rack travel cover;

FIG. 17 illustrates the disinfecting tower rack travel case in an open, position;

FIG. 18 is an illustrative embodiment of a wiring schematic;

FIG. 19 illustrates the disinfecting tower rack with an alternative UV light panel;

FIG. 20 is an illustration of the UV light panel used with the disinfecting tower rack shown in FIG. 18;

FIG. 21 illustrates an embodiment of the disinfecting tower rack having three UV light panels;

FIG. 22 illustrates the disinfection times of various distances from the UV light tower, UV dose (240 J/M²); and

FIG. 23 illustrates the disinfection times of various distances from the UV light tower, UV dose (120 J/M²).

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention is directed towards a disinfecting device, referred to generally as an Ultraviolet (UV) light tower 10, for use in disinfecting surfaces and the air associated with an area or enclosed room. The UV light tower 10 is designed to be 1) easily moved from one area to another within a structur, 2) easily transported from one facility to a second, independent, differently located facility, and 3) easily assembled and disassembled. The UV light tower 10 may be made from any material for maintaining its functionality, such as a matte black or white construction, with an unpainted surgical grade stainless steel reflector for maximum reflectance. The UV light tower 10 may also include an anti-microbial paint finish. The UV light, tower 10 may be powered using an electrical power cord for connection to an electrical outlet, or may be powered by a battery.

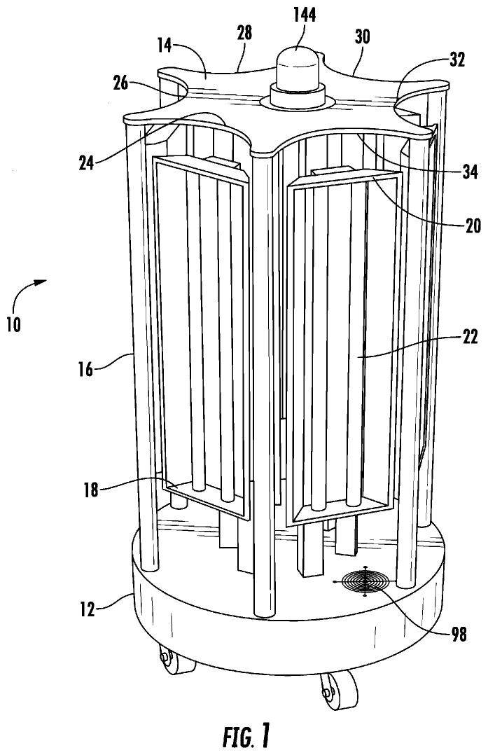

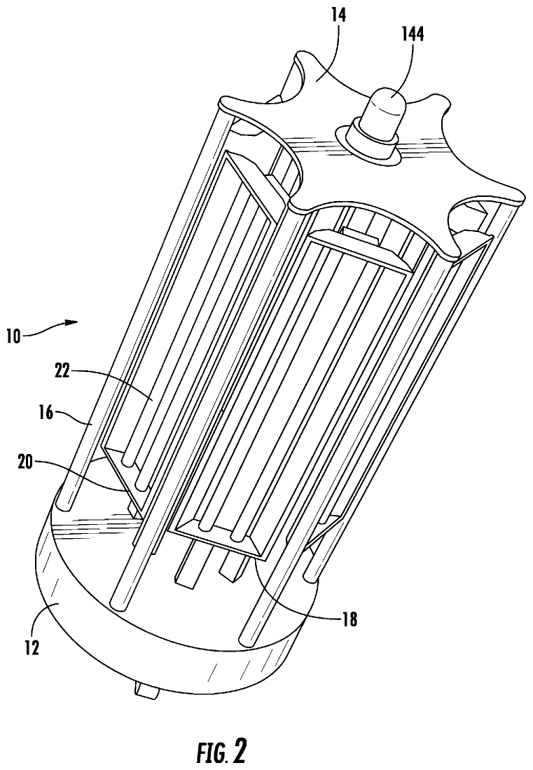

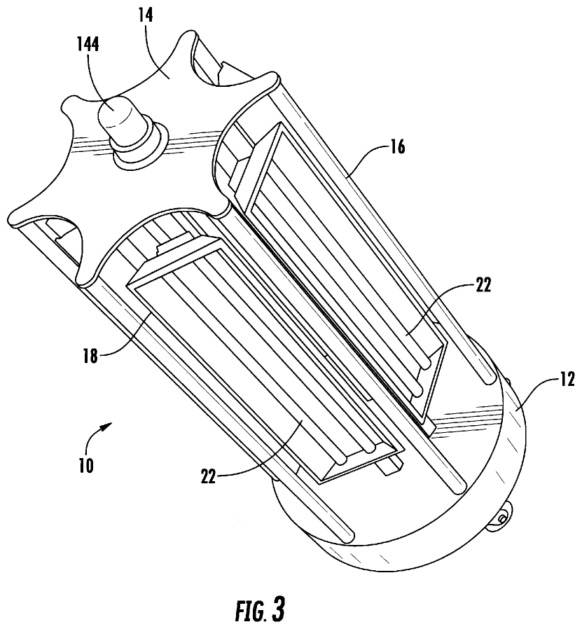

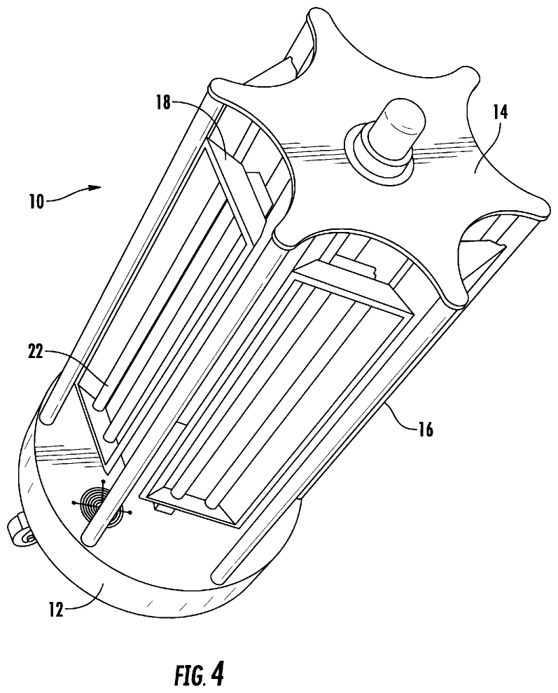

Referring to FIGS. 1-4, the UV light tower 10 is shown having a base 12 and upper plate 14, and a main body 16 separating the base 12 and the upper plate 14, thus defining an overall height. The base 12, the upper plate 14, and the main body 16 may be made of metal coated with a highly reflective coating. The UV light tower main body 16 is configured to receive and hold or secure to one or more UV light panels 18 (may also be referred to as UV light fixtures). The UV light tower 10 is shown utilizing six UV panels 18 to provide for complete 360° UVC disinfection. Preferably, the UV light tower 10 is configured so that when powered on, or turned on, each of the one or more UV light panels 18 provide UV light at the same time.

Each UV light panel 18 may be operationally separate and distinct from adjacent or other UV light panels 18. As such, each of one or more UV light panels 18 is configured to work independently (and function as a standalone UV light fixture), allowing each panel to be removed without impacting the functioning of any of the other secured or intact UV light panels 18. Each individual UV light panel 18 may be configured to be separately mountable to the UV light tower 10. Such functionality allows a user to add or remove the UV light panels 18 as needed. Each UV light panel 13 may be removably secured to the UV light tower 10 via a universal, adjustable yoke for use with many types of clamp options. Preferably, the UV light panels 18 comprise an outer case 20 having one or more LED UV bulbs or LEDs 22, preferably UVC bulbs or UVC LEDs (emitting in the range of 200-400 nm, preferably in the range of approximately 254 nm to about 280 nm). Since UV-C light is invisible to the human eye, each of the UV lights 22 may contain, a fluorescent phosphorous additive that illuminates visible light to ensure a user knows that the lamp is functional. While described as using UVC light, other UV lights that have the ability to inactivate pathogens (both viruses and bacteria) by impacting the cellular RNA and DNA, damaging nucleic acids, and preventing microorganisms from infecting and reproducing, or acting as a disinfectant may be used as well.

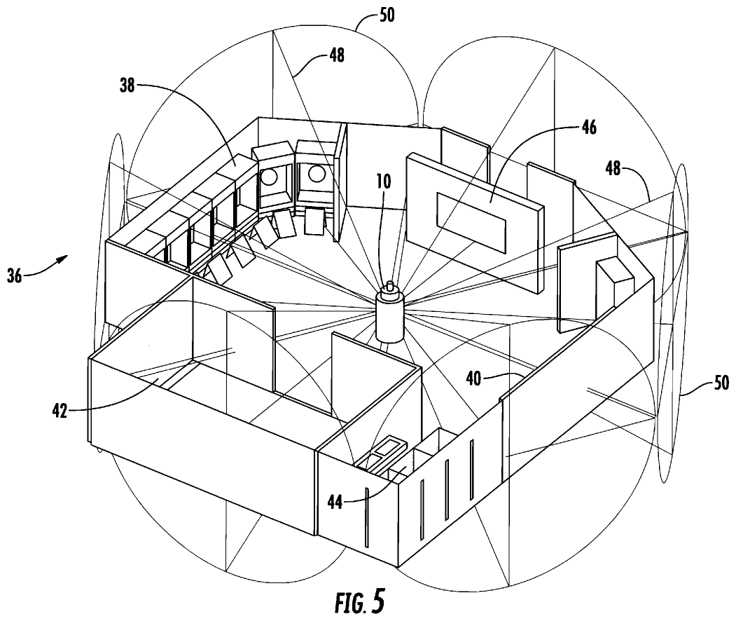

As illustrated in the figures, the UV light tower main body 16 is illustrated as having six (6) sides: a first side 24, a second side 26, a third side 28, a fourth side 30, a fifth side 32, and a sixth side 34. Each side is configured to receive and secure a UV light panel 18. While the UV light tower 10 is illustrated with a main body 16 having six sides, such embodiment is illustrative only. The UV light tower main body 16 may be designed to have more than six sides, such as eight or ten, or less than six sides, such as three, four or five. In any case, the UV light panels 18 are preferably positioned on each side or around the entire circumference of the UV light tower 10 so as to provide 360 degrees of UV light exposure. Referring to FIG. 5, the UV light, tower 10 is shown placed in a locker room 36. The 360-degree exposure of UV light is illustrated. Because the UV light panels 18 are around the circumference or at each side, 24, 26, 28, 30, 32, and 34, of the main body 16, the locker room is exposed to UV light in 360 degrees relative to the positioning of the UV light tower 10. As such, in operation, the lockers 38 and 40 and surrounding area, the showers 42 and surrounding area, the bathroom 44 and surrounding area, and the entrance way 46 and surrounding area, are simultaneously exposed to UV light, illustrated as lines 48 and circles 50. Although the locker room 36 is shown with the entire room being exposed to UV light, because the UV light tower 10 is configured to utilize removable UV light panels 18, one or more sections of the locker room 36 may not be exposed to UV simply by removing one or more of the UV light panels 18 from one or more sides of the main body 16.

Referring to FIG. 6, the UV light tower 10 is shown with each of the six UV light panels 18 removed. The UV light tower main body 16 comprises an outer frame 52 and an inner frame 54. The outer frame 52 comprises multiple outer frame members 56 secured to the base 12 near or along the outer edge 58 and spaced apart so as to foe positioned along the perimeter of the base 12. Each outer frame member 56 comprises a first end 60 sized and shaped to engage with or secure to the upper plate 14, a second end 62 sized and shaped to engage with or secure to the base 12, and a main body 64 separating the first end 60 and the second end 62. The inner frame 54 is configured to secure each of the UV light panels 18 thereto.

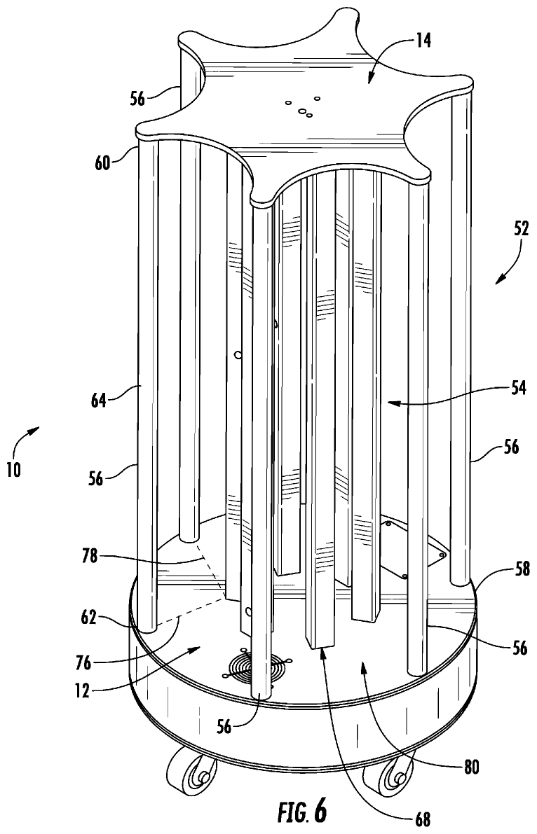

The inner frame 54 comprises multiple inner frame members 68, see also FIG. 7, secured near or towards the center of the base 12 and spaced apart from each other, and arranged to form a circular pattern. Each of the inner frame members 68 are also spaced apart from each of the outer frame members 56. Each inner frame member 68 comprises a first end 70 sized and shaped to engage with or secure to the upper plate 14, a second end 72 sized and shaped to engage with or secure to the base 12, and a main body 74 separating the first end 70 and the second end 72. The inner frame member main body 74 may have a hollow interior 76 for receiving and holding therein any cords or hardware necessary to operate the UV light panels 18. Each UV light panel 18 may be secured to an inner frame member main body 74 via, for example, mechanical fastening mechanisms such as screws or bolts screwed into taps in the inner frame members 68, or may be designed to be frictionally fit or snapped onto. Referring back to FIG. 6, the outer frame members 56 and the inner frame members 68 are separated by space or distance, 76, 78. One inner frame member 68 is positioned in between outer frame members 56. This arrangement, with the space or distance 76, 78, defines a UV light panel pocket 80. The UV light panel pocket 80 is sufficient in size to allow the UV light panel 18 to rest within.

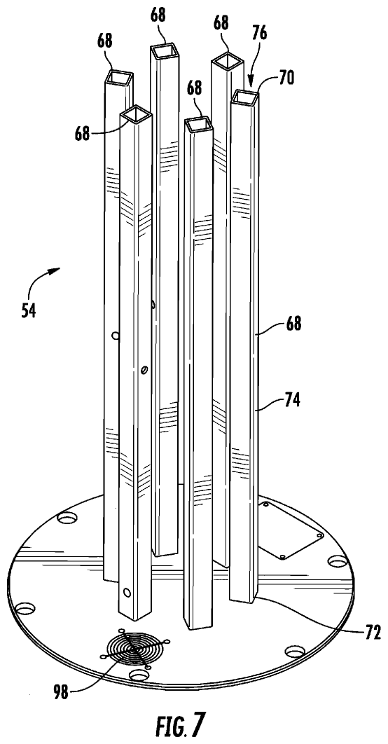

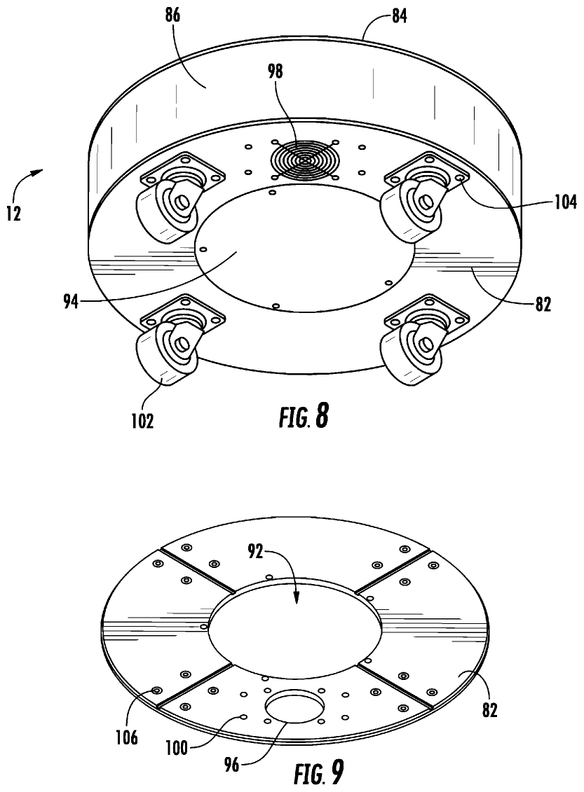

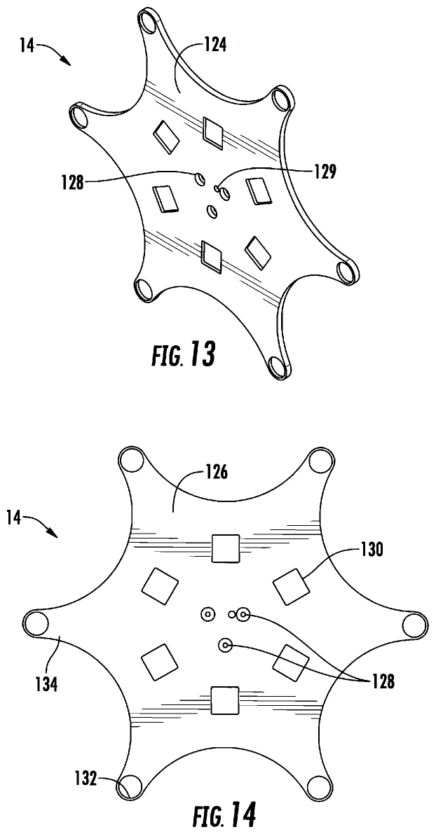

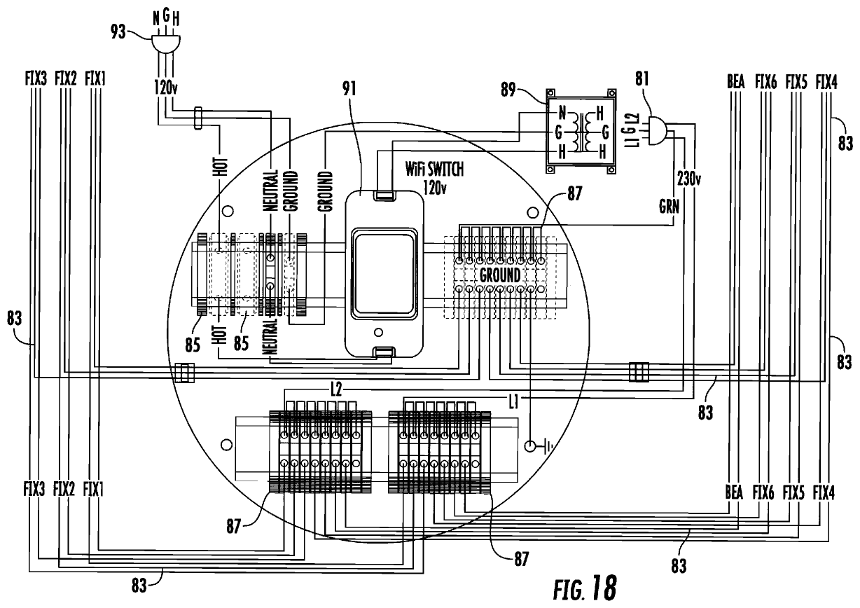

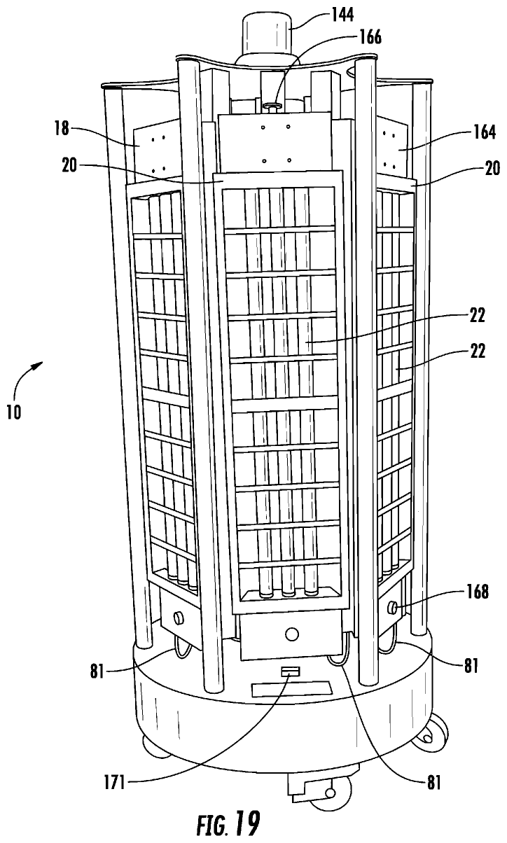

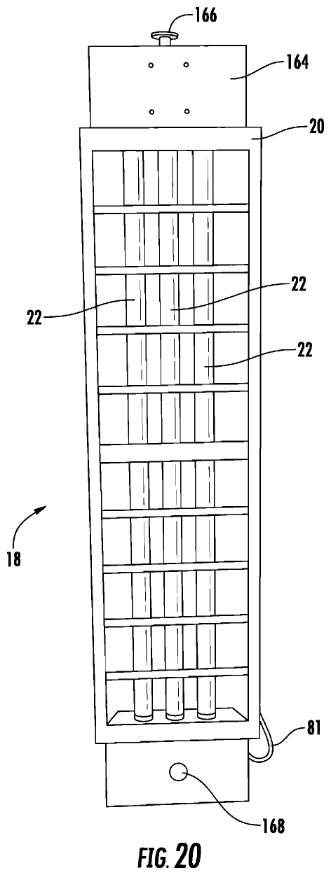

Referring to FIGS. 8-12, the base 12 is shown comprising a lower plate 82, an upper plate 84, and a continuous side wall 86 separating the lower plate 82 and the tipper plate 84. When engaged, the base plate 12 maintains the outer frame 52 and inner frame 54 in place. The interior 88 of the base 12 may include one or more spacers 90. The lower plate 82 may have a center opening 92 which may be covered by a removable plate 94 (secured thereto by screws), thus completely sealing off the interior 88. The interior 88 may house one or more components that are used to drive one or more functions associated with the UV light tower 10, such as UV light panels power cords 81, see FIGS. 18 and 19, wiring 83 for each of the UV light panels 18, referred to in the figure as “FIX” with a number to identify which of the one to six UV light panels 18 or the LED beacon in-use warning light 144, referred to as “BEA” in the figure, one or more fuses 85, one or more terminal blocks 87, and a transformer 89. The UV light tower 10 may include a WIFI switch 91 (with power cord 93) to be used to turn all UV light panels 18, “ON/OFF” via an app. Alternatively, the UV light tower 10 may be RF (radio frequency) compatible and include an RF key fob for quick on/off functioning. The UV tower 10 may be configured to operate using other wireless technologies, such as BLUETOOTH.

The UV tower 10 may be configured to be a smart device, and include the necessary hardware and/or software. The UV tower 10 may be configured to communicate with an electronic device, such as a smart phone or computer tablet, i.e. IPAD. Control of the UV light functioning, i.e. on/off, scheduling of on/off times, run times, when each of the individual UV light panels 18 turns on/off may also be accomplished using a cell phone. If needed, the UV tower 10 may include a control panel (including a processor, memory, and other hardware or software) with display operatively connected to one or more components or structures affecting functionality, allowing the user to turn on/off UV lights, turn off audio or visual alarms, or program on/off times or cycles by touch screen.

The lower plate 82 may contain an air flow opening 96, which may be covered by a grill 98 and one or more smaller ventilation openings 100. To aid in the portability of the UV light tower 10, one or more wheel assemblies, illustrated herein as castor wheels 102 may be secured to the lower plate 82 via screws 104 secured to openings 106.

The upper plate 84 may contain a center opening, which in FIG. 10 is covered by plate 108. Plate 108 may contain one or more openings 110, which allow tack welding of the spacers 90 to plate 108. The upper plate 84 may contain a plurality of openings. The first upper plate opening is a fan opening 112. Spaced about the outer edge 114. are a second set of openings, a plurality of outer frame member receiving openings 116, each sized and shaped to receive and hold outer frame members 56. The plurality of outer frame member receiving openings 116 are oriented in the same manner as that of the outer frame members 56. Towards the center of the upper plate 84 are a plurality of inner frame member receiving openings 118, each sized and shaped to receive and hold outer frame members 56. The plurality of inner frame member receiving openings 118 are oriented in the same manner as that of the inner frame members 68. An inner channel 120 sized and shaped to receive the vertical brace prior to welding may connect to one or more of the outer frame members receiving openings 116. An inner channel 122 sized and shaped to receive the vertical brace prior to welding may connect to one or more of the outer frame member receiving openings 116.

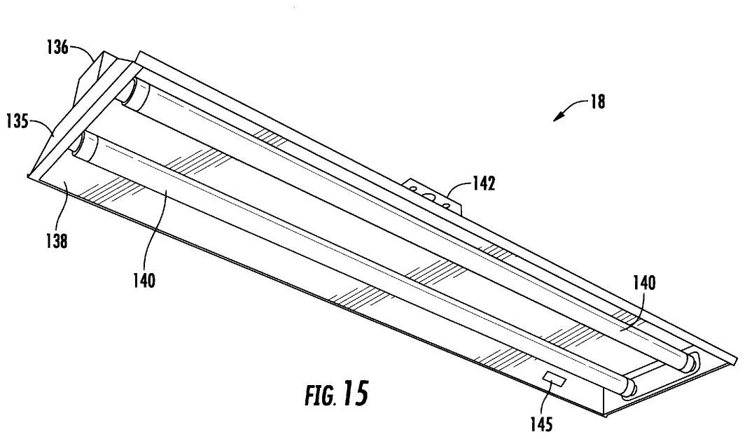

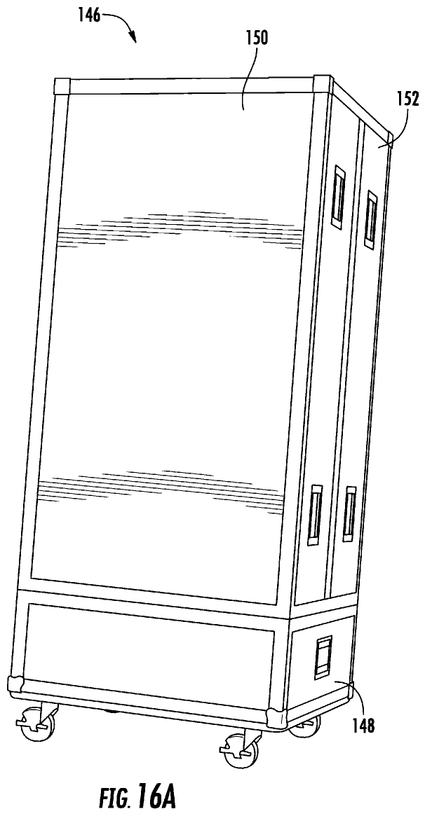

Referring to FIGS. 13 and 14, the upper plate 14 is shown having an inner surface 124 and an outer surface 126. The upper plate 14 may contain a first set of centrally positioned openings 128, sized and shaped for mounting the red beacon in-use warning system 144 (see FIG. 1 to be described later. The three centrally positioned openings 128 are for the bolts associated with the red beacon in-use warning system 144 and secured with a nut. A center opening 129 is provided for the power cable 81 which runs over to the inner frame member 68, where a cable gland near the top may be provided. Spaced from the openings 128 are a second set of openings, a plurality of inner frame member receiving openings 130, each sized and shaped to receive and hold an inner frame member 68. The plurality of inner frame member receiving openings 130 are oriented in the same manner as that of the inner frame members 68. Spaced from the plurality of inner frame member receiving openings 130 are a third set of openings, a plurality of outer frame member receiving openings 132, each sized and shaped to receive and hold outer frame members 56. The plurality of outer frame member receiving openings 132 may be located at the tip of the finger like structures 134 associated with the hexagonal, scalloping form of the upper plate 14. The plurality of outer frame member receiving openings 132 are oriented in the same manner as that of the outer frame members 58. When engaged, the upper plate 14 maintains the outer frame 52 and inner frame 54 in place.

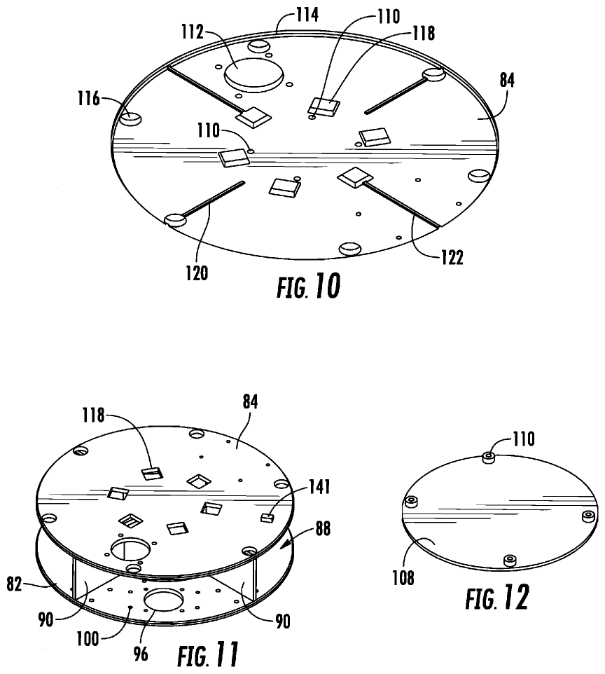

FIG. 15 provides an illustrative example of the UV light panel 18. The UV light panel 18 may include a source housing 135, an electrical housing 136, which may include the electrical hardware and ballast (not shown), a reflector 138, a UV radiator or lamp 140 and a bracket 142 for securing an inner frame member main body 74. The multiple UV light panels 18 are preferably operatively and electrically connected together so that when the UV light tower 10 is powered on (either directly when UV light tower 10 power cord is plugged into an electrical outlet, or when a power ON/OFF button 141, see FIG. 11, is pressed), the individual UV light panels 18 provide UV light at the same time. In an illustrative embodiment, each of the individual UV light panel power cords 81 may be plugged into a central power outlet bank, allowing each to turn on/off simultaneously when the UV light tower 10 is turned on/off. All the electrical hardware required, including a power cord(s), electrical outlet receivers, and other components for functionality, such as processors or microchips with memory, wireless technology such as a WIFI/RF/BLUETOOTH receivers and/or transmitters, may be placed within the base 20.

To prevent accidental UV light exposure, the UV light tower 10 may include an in-use warning system. The in-use warning system may include an LED beacon in-use warning light 144, see FIG. 1. When in use and the UV light tower 10 is exposing air and/or surfaces to UVC light, the LED beacon in-use warning light 144 is turned on automatically. As soon as the UV light is turned off, the LED beacon in-use warning light 144 automatically turns off. The LED beacon in-use warning light 144 may be configured to flash and have an audio alarm. The UV light tower 10 may include one or more occupancy sensor(s) 145, see FIG. 15, such as motion sensors or heat/thermal sensors, within the. disinfection range (air or surface, or area being exposed to the UV light) configured to immediately shut off the UV light tower 10 if a person or animal is within the disinfection range while the UV light tower 10 is in the “0N” or operating position, thus preventing accidental human/animal UV light exposure when the UV lights are operating. While the sensor 145 may be part of or integrated into the one or more UV light panels 18, the sensor 145 may additionally or alternatively be incorporated into the UV light tower 10, such as the base 12, upper plate 14, main body 16, or as part of the UV light bulbs 22. In one embodiment, each of the one or more UV light panels 18 may include its own occupancy sensors. All of the one or more UV light panels may be turned off simultaneously, or each individual one or more UV light panels 18 may be turned off independently relative to each other.

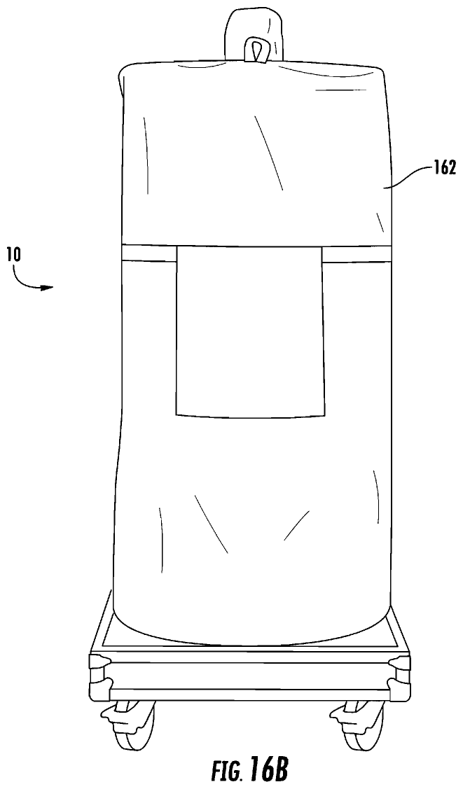

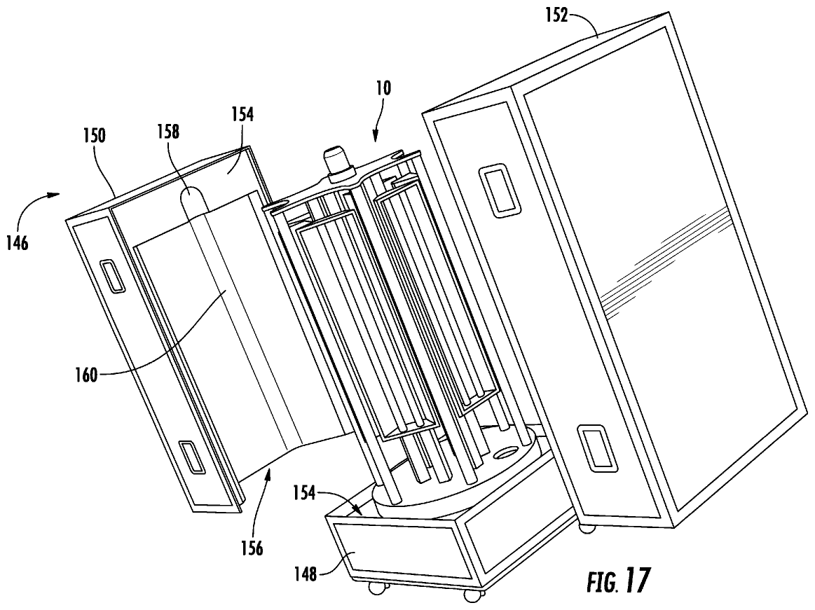

To aid in portability, the UV light tower 10 may be stored and transported in a UV light tower storage, unit 146, see FIG. 16A and FIG. 17. The UV light tower storage unit 146 comprises a bottom portion 148 and two side portions 150 and 152, which, when secured together, enclose the UV light tower 10. The bottom portion 148 has an interior 154 sized and shaped to hold the base 12. Each of the two side portions 150 and 152 has an interior region sized and shaped to cover portions of the UV light tower 10. Optionally, the two side portions 150 and 152 may contain a cushioning 154, such as foam, to prevent movement. The cushioning 154 may contain cutouts 158 and 160 in the shape of the LED beacon in-use warning light 144 and outer frame members 56 to prevent movement of the UV light tower 10 when stored and locked in the UV light tower storage unit 146.

FIG. 16B illustrates the UV light tower 10 with a cover 162, such as a vinyl material.

Referring to FIG. 19, the UV light tower 10 is shown with an alternative embodiment of the one or more UV panels 18, The outer case 20 of the UV light panels 18 is shown having three LED UVC bulbs 22 (emitting in the range of 200-400 nm, preferably in the range of approximately 254 nm to about 280 nm), having a 180-degree field of UVC radiation. The outer case 20 may be secured to a bracket or plate 164, thus allowing a user to remove from or add to each of the one or more UV light panels 18 separately and independently. As described before, each of the one or more UV light panels 18 are configured to act as a standalone UV light fixture or fit into the UV light tower 10. The bracket or plate 164 may include one or more handles 166 to allow a user to adjust the positioning of the one or more UV light panels 18 about the UV light tower 10. Each of the one or more UV panels 18 may have a True1 connection with True1 pass-through, allowing the ability to link multiple units per designated circuit. The one or more UV panels 18 may also each include its own light ON/OFF indicator 168.

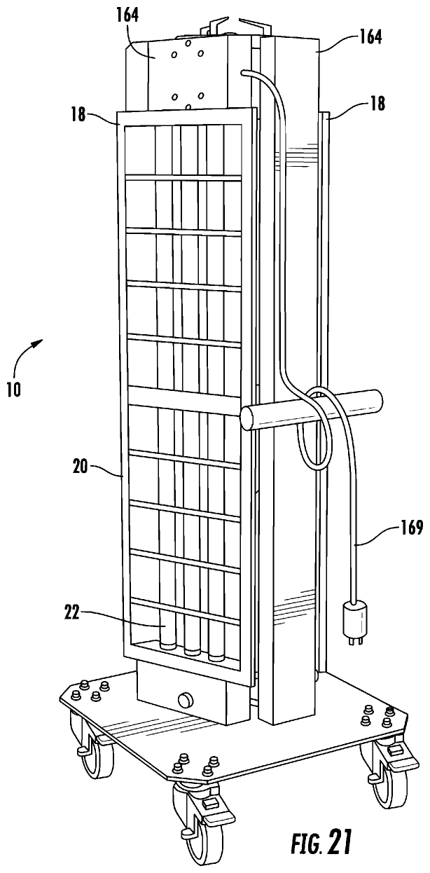

While the UV light tower 10 is shown as a six UV panels 18 tower, such number of UV panels 18 is illustrative only. The UV light tower 10 may include more UV light panels 18 or less UV light panels 18. Referring to FIG. 21, a “mini” UV light tower 10 is illustrated. The “mini” UV light tower 10 is designed to utilize three UV light panels 18. The “mini” UV light tower 10 is preferably configured as described above. The multiple UV light panels IS are preferably operatively and electrically connected together so that when the mini UV light tower 10 is powered on (either directly when mini UV light tower 10 power cord 169 is plugged into an electrical outlet, or when a power ON/OFF button 171 (see FIG. 19), is pressed), each individual UV light panel 18 provides UV light at the same time.

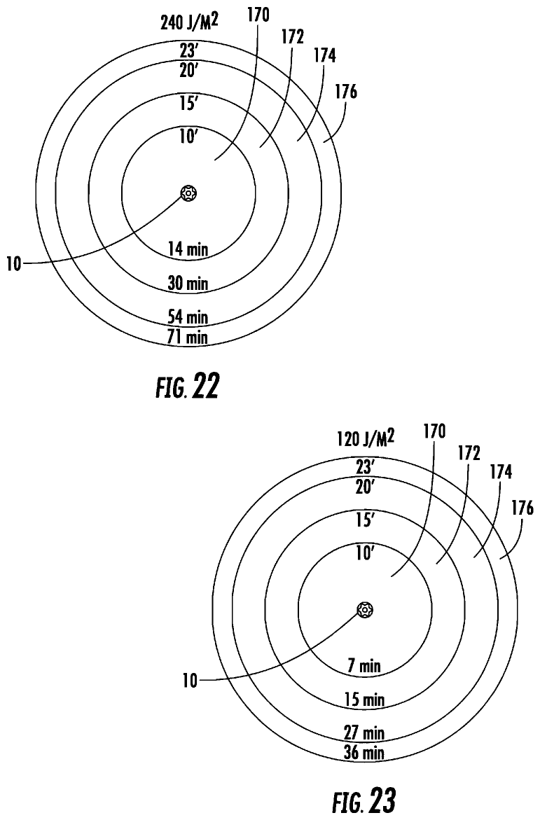

Referring to FIGS. 22 (240 J/M²) and 23 (120 J/M²), the UV light tower 10 may be designed to include UV light exposure to provide 360° disinfection to disinfect exposed areas in a certain time period per distance from the where the UV light tower 10 is positioned in a room or area to be disinfected. Depending on the UV dose, the UV light tower 10 is designed to have 360° exposure disinfect kill times based on the distance from the UV light tower 10, see 1) 360° disinfection ring 170, 10 feet from the UV light tower 10 having disinfection time of 14 minutes; 2) 360° disinfection ring 172, 15 feet from the UV light tower 10 having disinfection time of 30 minutes; 3) 360° disinfection ring 174, 20 feet from the UV light tower 10 having disinfection time of 54 minutes; and 4) 360° disinfection ring 176, greater than 20 feet from the UV light tower 10 having disinfection time of 72 minutes.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is;

1. A disinfecting light tower comprising;
a plurality of light panels having a light source configured to disinfect a surface or air,
a support structure configured to house said one or more light panels.

2. The disinfecting light tower according to claim 1, wherein each of said light panels are removably secured to said support structure.

3. The disinfecting light tower according to claim 1, wherein said light source is one or more UVC lights.

4. The disinfecting light tower according to claim 1, wherein said plurality of light panels are configured to produce light independently or together as a unit when secured to said disinfecting light tower.

5. The disinfecting light tower according to claim 1, wherein the number of said plurality of light panels is sufficient to provide a 360-degree field of radiation.

6. The disinfecting light tower according to claim 1, wherein said plurality of light panels are arranged to provide adjacent light panels with overlapping fields of light radiation.

7. The disinfecting light tower according to claim 1, further including a visual warning device.

8. The disinfecting light tower according to claim 1, further including an audio safety warning device.

9. The disinfecting light tower according to claim 1, wherein said disinfecting light tower is configured to stop operating when a person or animal enters a field of radiation.

10. The UV light disinfecting tower for use in disinfecting surfaces or the air associated with an area or enclosed room comprising:
a base
an upper plate, and
a main body, said main body having a plurality of UV light panels secured thereupon, said UVC light panels comprising one or mere UVC lights

11. The UV light disinfecting tower for use in disinfecting surfaces or the air associated with an area or enclosed room according to claim 10, wherein each of said UVC light panels are removably secured to said main body.

12. The UV light disinfecting tower for use in disinfecting surfaces or the air associated with an area or enclosed room according to claim 10, wherein said plurality of UVC light panels are configured to produce light independently or together as a unit when secured to said disinfecting light tower.

13. The UV light disinfecting tower for use in disinfecting surfaces or the air associated with an area or enclosed room according to claim 10, wherein said UVC light panels are arranged about said main body in an orientation to provide a 360-degree field of UVC radiation.

14. The UV light disinfecting tower for use in disinfecting surfaces or the air associated with an area or enclosed room according to claim 10, wherein said main body comprises an inner frame configured to secure each of said UV light panels.

15. The UV light disinfecting tower for use in disinfecting surfaces or the air associated with an area or enclosed room according to claim 10, further including a warning light panels.

16. The UV light disinfecting tower for use in disinfecting surfaces or the air associated with an area or enclosed room according to claim 10, further including an audio alarm.

17. The UV light disinfecting tower for use in disinfecting surfaces or the air associated with an area or enclosed room according to claim 10, further including one or more occupancy sensors configured to immediately shut off said UV light disinfecting tower if a person or animal is within a disinfection range while said UV light disinfecting tower is in operating position.

18. The UV light disinfecting tower for use in disinfecting surfaces or the air associated with an area or enclosed room according to claim 10, wherein said tower is configured to operate via wireless technology.

19. The UV light disinfecting tower for use in disinfecting surfaces or the air associated with an area or enclosed room according to claim 10, wherein said tower is configured to communicate with a smart phone or a computer tablet.
